(19) United States
(12) Plant Patent Application Publication
(10) Pub. No.: US 2003/0196236 P1
(43) Pub. Date: Oct. 16, 2003

(54) Chrysanthemum plant named 'Chatry'

(76) Inventor: Jean-Pierre Challet, Nauille (FR)

Correspondence Address: BURNS DOANE SWECKER & MATHIS L L P, POST OFFICE BOX 1404, ALEXANDRIA, VA, 22313-1404, US

(21) Appl. No.: 10/119,898

(22) Filed: Apr. 11, 2002

(51) Int. Cl. A01H 5/00

(52) U.S. Cl. PLT/288

(57) ABSTRACT

A new and distinct Chrysanthemum cultivar is provided that is a mutation of the ‘Chalotari’ cultivar (non-patented in the United States). Attractive ball-shaped white blossoms at full maturity are formed in profusion. Such blossoms commonly are approximately 13 cm in diameter when fully open. The growth habit is rustic and very compact and the plant displays good branching following pinching. The foliage is dark green and contrasts well with the white blossoms. The stems are short and strong. A response time of approximately ten weeks is exhibited. The plant grows well in pots.

### BOTANICAL/COMMERCIAL CLASSIFICATION

Dendranthema grandifloria/Decorative Pot Mum

### VARIETAL DENOMINATION

cv. ‘Chatry’

### SUMMARY OF THE INVENTION

The present invention comprises a new and distinct cultivar of Chrysanthemum, botanically known as Dendranthema grandiflora, and hereafter is referred to by the cultivar name ‘Chatry’.

The new cultivar of the present invention was created at Nuaillé, France when plants of the ‘Chalotari’ cultivar (non-patented in the United States) were irradiated with gamma radiation. Cuttings of the irradiated plants were used to produce new plants that were carefully studied. The new cultivar of the present invention was selected from among the rooted cuttings at Nuaillé, France, and displays a distinctive and attractive flower coloration.

It was found that the new Chrysanthemum cultivar of the present invention displays:

(a) a very compact rustic growth habit with good branching following pinching,

(b) forms attractive ball-shaped long-lasting decorative white blossoms when fully mature,

(c) forms short strong stems,

(d) forms attractive luxuriant dark green leaves, and

(e) an ability to grow well in pots.

The new cultivar can be grown singly or in clumps in pots. It also can be grown in the landscape. The bright white blossoms when fully mature contrast nicely with the dark green foliage. The blossoms have been found to be long lasting and commonly last up to four weeks on the plant. The plant branches well following pinching.

The new cultivar can be readily distinguished from its ‘Chalotari’ parent in view of its shorter stature and different blossom coloration. The blossoms of the cultivar of the present invention are a white when fully mature while those of the ‘Chalotari’ cultivar are pink in coloration.

The ‘Chatour’ cultivar (U.S. Plant patent application No. ______, filed concurrently herewith) is a further mutation formed through the irradiation of the ‘Chalotari’ cultivar and displays distinctive mauve-colored blossoms.

Asexual reproduction of the new cultivar by cuttings as performed at Nuaillé, France, in a controlled environment has demonstrated that the characteristics of the new cultivar are firmly fixed and are retained through successive generations of asexual propagation.

‘Chatry’ has not been observed under all possible environmental conditions to date. Accordingly, it is possible that the phenotype may vary somewhat with variations in the environment, such as temperature, light, day length, contact with pesticides and/or subjection to grown regulation treatments.

### BRIEF DESCRIPTION OF THE PHOTOGRAPH

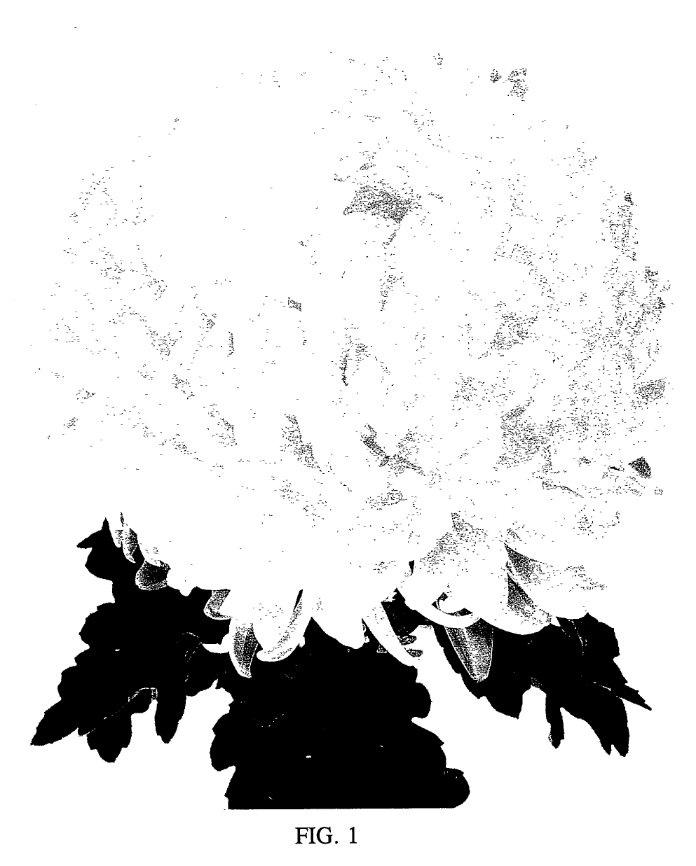

The accompanying FIG. 1 depicts a typical blossom when fully mature and the dark green foliage of the new ‘Chatry’ cultivar while growing in a pot in a greenhouse at Nuaillé, France. The configuration of the attractive ball-shaped white blossom is illustrated.

### DETAILED DESCRIPTION

The plants described were grown in pots in a greenhouse at Nuaillé, France. No growth regulation was used. The growing conditions approximate those commonly utilized for the commercial production of decorative pot mums.

General Appearance:

Very compact with a very rustic growth habit.

The overall plant is sorter than that of the ‘Chalotari’ cultivar.

Foliage:

Attractive dark green. Such foliage is abundant and vigorous.

Stems:

Short and strong.

Flowers:

The blossoms are initially of a creamy coloration with a darker center and change to white with full maturity. In contrast the ‘Chalotari’ parent cultivar forms pink blossoms and the ‘Chatour’ cultivar forms darker mauve blossoms. The configuration is generally ball-shaped and the diameter is approximately 13 cm when fully expanded.

Flower Duration:

Very long lasting with the blossoms commonly lasting up to approximately four weeks on the plant.

Response Time:

The time between the beginning of the short day period and the flowering date at normal temperature and light intensity is approximately ten weeks.

Usage:

Decorative pot mum.

I claim:

1. A new and distinct cultivar of Chrysanthemum plant that displays:
(a) a very compact rustic growth habit with good branching following pinching,
(b) forms attractive ball-shaped long-lasting decorative white blossoms when fully mature,
(c) forms short strong stems,
(d) forms attractive luxuriant dark green leaves,
(e) an ability to grow well in pots;
substantially as illustrated and described.
